(19) United States
(12) Patent Application Publication
(10) Pub. No.: US 2008/0195188 A1
(43) Pub. Date: Aug. 14, 2008

(54) IMPLANTABLE MEDICAL DEVICE WITH FIXATION MECHANISM

(75) Inventors: Imad Libbus, St. Paul, MN (US); Randy Westlund, River Falls, WI (US)

Correspondence Address: SCHWEGMAN, LUNDBERG & WOESSNER, P.A., P.O. BOX 2938, MINNEAPOLIS, MN, 55402, US

(73) Assignee: CARDIAC PACEMAKERS, INC., ST. PAUL, MN (US)

(21) Appl. No.: 11/673,186

(22) Filed: Feb. 9, 2007

(51) Int. Cl. A61N 1/05

(52) U.S. Cl. 607/118; 607/149

(57) ABSTRACT

Miniature implantable medical devices each include a fixation mechanism to prevent the device from migrating in the body of a patient after implantation. The fixation mechanism is activated after the device is positioned in the body. In one embodiment, the miniature implantable medical devices each include a circuit to deliver neurostimulation. In one embodiment, the fixation mechanism includes one or more externally activated anchoring devices each configured to switch from a substantially non-anchoring state to a substantially anchoring state in response to an externally applied energy. In another embodiment, implantable medical devices are magnetically attracted to each other to prevent the devices from migrating in the body after implantation.

### CROSS-REFERENCE TO RELATED APPLICATION

This application is related to co-pending, commonly assigned U.S. patent application Ser. No. 11/548,354, entitled “IMPLANTABLE NEUROSTIMULATOR FOR MODULATING CARDIOVASCULAR FUNCTION,” filed on Oct. 11, 2006, which is hereby incorporated by reference its entirety.

### TECHNICAL FIELD

This document relates generally to implantable medical devices and particularly to implantable medical devices with a fixation mechanism.

### BACKGROUND

Neurostimulation has been applied or proposed to modulate various physiologic functions and treat various diseases. One example is the modulation of cardiovascular functions by stimulating sympathetic and parasympathetic nerves that innervate the heart. Activities in the vagus nerve, including artificially applied electrical stimuli, modulate the heart rate and contractility (strength of the myocardial contractions). Electrical stimulation applied to the vagus nerve is known to decrease the heart rate and the contractility, lengthening the diastolic phase of a cardiac cycle. This ability of the vagal nerve stimulation may be utilized, for example, to control myocardial remodeling. Electrical stimulation applied at acupuncture points is also known to have therapeutic effects in cardiovascular functions.

Neurostimulation provides therapeutic benefit when applied shortly after the occurrence of a cardiac disorder event such as acute myocardial infarction (MI). For example, after the acute MI, adverse ventricular remodeling starts and the heart is more susceptible to arrhythmias. Neurostimulation may be applied to control the post-MI ventricular remodeling and prevent the arrhythmias from occurring. For prompt deployment of a neurostimulation system following a cardiac disorder event such as acute MI, and for other reasons, there is a need for a neurostimulator that is implantable using a minimally invasive procedure.

### SUMMARY

Miniature implantable medical devices each include a fixation mechanism to prevent the device from migrating in the body of a patient after implantation. The fixation mechanism is activated after the device is positioned in the body. In one embodiment, the miniature implantable medical devices each include a circuit to deliver neurostimulation.

In one embodiment, a system includes an implantable medical device and an external anchoring activator. The implantable medical device includes electronic circuitry, an implantable housing including a wall forming a chamber to house the electronic circuitry, and one or more externally activated anchoring devices coupled to the implantable housing. The one or more externally activated anchoring devices are each configured to switch from a substantially non-anchoring state to a substantially anchoring state in response to an externally applied energy. The external anchoring activator is configured to be operably positioned with respect to the implantable medical device to provide the externally applied energy.

In another embodiment, an implantable system includes a plurality of magnetically positionable implantable medical devices with positioning elements configured to prevent the devices from migrating in a body using magnetic attraction. The magnetically positionable implantable medical devices are magnetically attracted to each other after being placed in the body.

In one embodiment, a method for operating an implantable medical device in a body is provided. An energy applied externally to the body is received. In response, one or more externally activated anchoring devices of the implantable medical device are switched from a substantially non-anchoring state to a substantially anchoring state.

In another embodiment, a method for operating a system of a plurality of implantable medical devices in a body is provided. Each of the implantable medical devices in the body is provided with a positioning element that includes a magnetized or ferromagnetic material. The implantable medical devices are prevented from migrating in the body by using magnetic attraction between these implantable medical devices.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 is an illustration of an embodiment of a neurostimulation system and portions of an environment in which the neurostimulation system is used.

FIG. 2 is an illustration of another embodiment of the neurostimulation system and portions of the environment in which the neurostimulation system is used.

FIG. 3 is an illustration of an embodiment of an implantable medical device of the neurostimulation system.

FIG. 4 is an illustration of another embodiment of the implantable medical device.

FIG. 5 is a block diagram illustrating an embodiment of a circuit of the implantable medical device.

FIGS. 6A-B are illustrations of an embodiment of an implantable medical device with an externally activated anchoring system.

FIGS. 7A-B are illustrations of another embodiment of an implantable medical device with an externally activated anchoring system.

FIGS. 8A-B are illustrations of another embodiment of an implantable medical device with an externally activated anchoring system.

FIGS. 9A-C are detailed illustrations of an embodiment of the externally activated anchoring system of FIGS. 8A-B.

FIGS. 10A-C are each an illustration of an embodiment of a magnetically positionable implantable medical device.

FIG. 11 is an illustration of an embodiment of an implantable medical device placed in a body lumen.

FIG. 12 is an illustration of an embodiment of a system of a plurality of magnetically positionable implantable medical devices.

FIG. 13 is a flow chart illustrating a method for externally activating an anchoring system of an implantable medical device.

FIG. 14 is a flow chart illustrating a method for magnetically coupling a plurality of implantable medical devices.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses miniature implantable medical devices each including a fixation mechanism that prevents or limits the implanted device from migrating in the body of a patient and/or a positioning element that allows controlled movement of the device during implantation. In one embodiment, the positioning element is used to restrict the movement of an implantable medical device implantation, thus functioning as the fixation mechanism. The miniature implantable medical devices each include an implantable housing containing a circuit that delivers electrical stimulation and/or senses a physiologic signal through electrodes. While miniature implantable neurostimulators (also known as microstimulators) are discussed in this document as a specific example, the present fixation and positioning methods and devices apply generally to any implantable medical devices that provide for therapeutic and/or monitoring functions. While modulation of cardiovascular functions is discussed in this document as a specific example of application of neurostimulation, the present subject matter also applies to implantable neurostimulators treating non-cardiovascular disorders such as epilepsy, depression, obesity, diabetes, pain, paralysis and other motor dysfunctions, and incontinence.

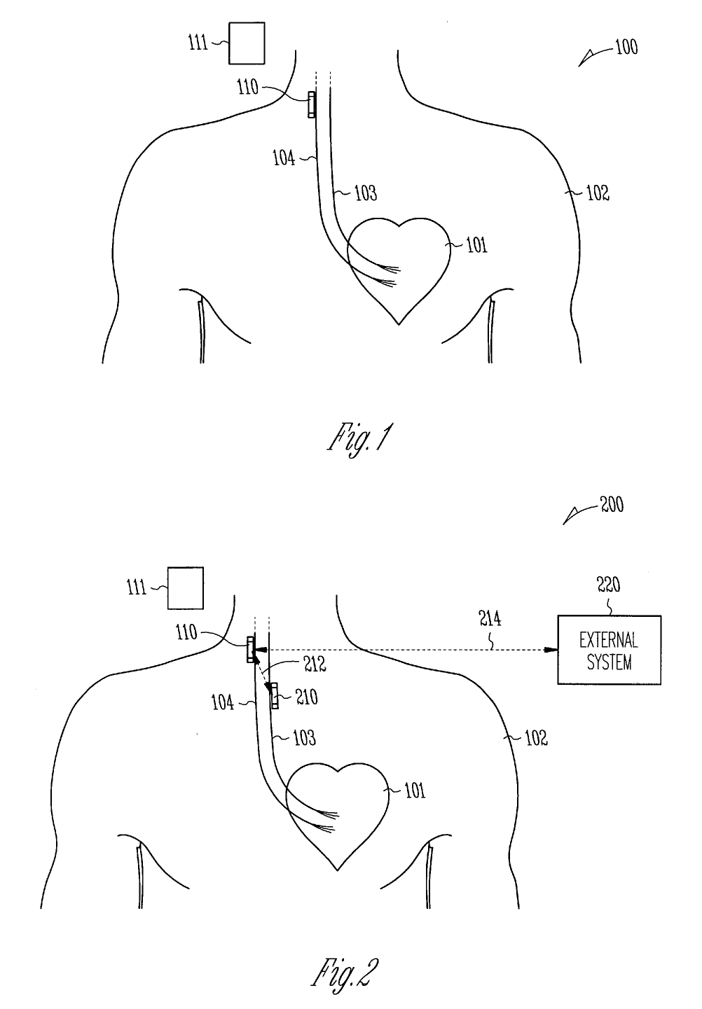

FIG. 1 is an illustration of an embodiment of a neurostimulation system 100 and portions of an environment in which system 100 is used. System 100 includes a miniature implantable medical device 110 that delivers neurostimulation to a body 102 having a heart 101 and nerves 103 and 104 that innervate heart 101. In one example for illustrative purposes only, nerve 103 represents a nerve of the sympathetic nervous system, and nerve 104 represents a nerve of the parasympathetic nervous system. In one embodiment, implantable medical device 110 delivers neurostimulation to any component of the nervous system whose activities affect one or more cardiovascular functions. Examples of such component of the nervous system include baroreceptors, aortic nerve, carotid nerve, vagus nerve, the spinal cord dorsal or ventral nerves, and the sympathetic ganglia and nerves.

Implantable medical device 110 delivers neurostimulation by executing a stimulation algorithm for modulating a cardiovascular function. In the illustrated embodiment, implantable medical device 110 is implanted in a vicinity of parasympathetic nerve 104. By controlling stimulation parameters, the neurostimulation is delivered to stimulate or inhibit parasympathetic nerve traffic, thus increasing or decreasing the parasympathetic tone. In one embodiment, implantable medical device 110 is also capable of sensing physiological signals, such as neural activities in nerve 104 or cardiac electric signals.

In various embodiments, implantable medical device 110 includes one or both of a positioning mechanism and a fixation mechanism. An external device 111 controls the positioning mechanism and/or the fixation mechanism when being operably positioned with respect to implantable medical device 110. After implantation, the positioning mechanism allows for adjustment of the location and/or orientation of implantable medical device 110 in body 102, and the fixation mechanism allows for anchoring of implantable medical device 110 to a portion of tissue in body 102. Various embodiments of the positioning mechanism and the fixation mechanism are discussed below.

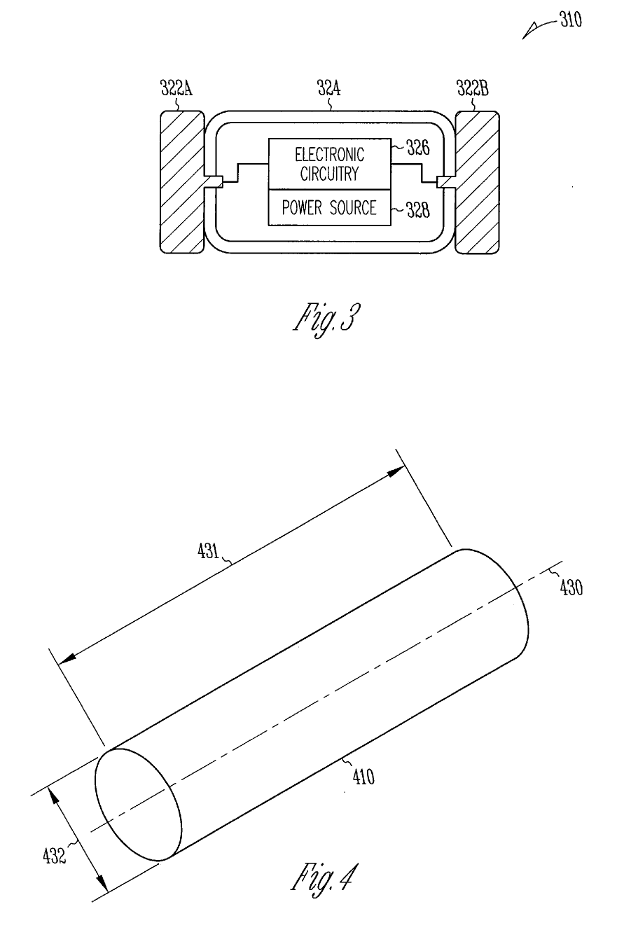

FIG. 2 is an illustration of an embodiment of a neurostimulation system 200 and portions of an environment in which system 200 is used. System 200 includes implantable medical device 110, another implantable medical device 210, an external system 220, a telemetry link 212 providing for communication between implantable medical devices 110 and 210, and another telemetry link 214 providing for communication between implantable medical devices 110 and external system 220.

Implantable medical device 210 includes a neurostimulation circuit and/or a sensing circuit. In one embodiment, implantable medical device 210 senses a physiological signal and transmits data to implantable medical device 110 via telemetry link 212 to allow implantable medical device 110 to use the sensed physiological signal to control the delivery of the neurostimulation. In one embodiment, implantable medical device 210 delivers neurostimulation by executing a stimulation algorithm for modulating a cardiovascular function. For example, as illustrated in FIG. 2, implantable medical device 110 is implanted in a vicinity of nerve 104, and implantable medical device 210 is implanted in a vicinity of nerve 103. By controlling stimulation parameters, parasympathetic stimulation is delivered from implantable medical device 110 to stimulate or inhibit parasympathetic nerve traffic, thus increasing or decreasing the parasympathetic tone, and sympathetic stimulation is delivered from implantable medical device 210 to stimulate or inhibit sympathetic nerve traffic, thus increasing or decreasing the sympathetic tone. In one embodiment, implantable medical devices 110 and 210 are coordinated using telemetry link 212 to control autonomic balance in body 102 by delivering one or more of parasympathetic excitation, parasympathetic inhibition, sympathetic excitation, and sympathetic inhibition.

In various embodiments, implantable medical device 210 includes one or both of a positioning mechanism and a fixation mechanism. In the illustrated embodiment, external device 111 also controls the positioning mechanism and/or the fixation mechanism when being operably positioned with respect to implantable medical device 210. After implantation, the positioning mechanism allows for adjustment of the location and/or orientation of implantable medical device 210 in body 102, and the fixation mechanism allows for anchoring of implantable medical device 210 to a portion of tissue in body 102. Various embodiments of the positioning mechanism and the fixation mechanism are discussed below.

Telemetry link 212 provides intra-body telemetry between implantable medical devices. In one embodiment, telemetry link 212 is a far-field radio-frequency (RF) telemetry link. In another embodiment, telemetry link 212 is an ultrasonic telemetry link.

External system 220 communicates with implantable medical device 110 via telemetry link 214, thus providing for access to implantable medical devices 110 and 210 by a physician or other caregiver. In the illustrated embodiment, implantable medical device 110 functions as a central implantable device in system 200 that coordinates the operation of implantable medical devices 210 via telemetry link 212. In another embodiment, an implantable medical device of a type different from implantable medical devices 110 or 210 is used as the central implantable device in system 200. Examples of such a central implantable device include known types of pacemakers, cardioverter/defibrillators, cardiac resynchronization devices, cardiac remodeling control devices, and any other implantable device capable of communicating with, and coordinating operation of, devices such as implantable medical devices 110 or 210. In one embodiment, external system 220 includes a programmer. In another embodiment, external system 220 is a patient management system including an external device communicating with implantable medical device 110 via telemetry link 214, a remote device in a relatively distant location, and a telecommunication network linking the external device and the remote device. The patient management system allows access to implantable medical devices 110 and 210 from a remote location, for purposes such as monitoring patient status and adjusting therapies. In one embodiment, telemetry link 214 is an inductive telemetry link. In another embodiment, telemetry link 214 is a far-field RF telemetry link. In another embodiment, telemetry link 214 is an ultrasonic telemetry link (with an external acoustic coupler attached to the surface of body 102 during a telemetry session to use tissue of body 102 as the media for acoustic signal transmission). Telemetry link 214 provides for data transmission from implantable medical device 110 to external system 220. This includes, for example, transmitting real-time physiological data acquired by implantable medical devices 110 and/or 210, extracting physiological data acquired by and stored in implantable medical devices 110 and/or 210, extracting patient history data such as occurrences of arrhythmias and therapy deliveries recorded in implantable medical devices 110 and/or 210, and/or extracting data indicating an operational status of implantable medical devices 110 and/or 210 (e.g., battery status). Telemetry link 214 also provides for data transmission from external system 220 to implantable medical devices 110 and/or 210. This includes, for example, programming implantable medical devices 110 and/or 210 to acquire physiological data, programming implantable medical devices 110 and/or 210 to perform at least one self-diagnostic test (such as for a device operational status), and/or programming implantable medical devices 110 and/or 210 to deliver neurostimulation and/or to adjust the delivery of the neurostimulation.

In various embodiments, system 200 includes a network of miniature implantable medical devices such as implantable medical devices 110 and 210 to modulate one or more cardiovascular functions using neurostimulation. The miniature implantable medical devices each deliver neurostimulation and/or sense a physiologic signal.

FIG. 3 is an illustration of an embodiment of a miniature implantable medical device 310, which represents a specific embodiment of implantable medical device 110 or implantable medical device 210. Implantable medical device 310 includes electronic circuitry 326, a power source 328, an implantable housing 324, and electrodes 322A-B. Electronic circuitry 326 delivers neurostimulation and/or senses a physiologic signal. Power source 328 supplies energy to electronic circuitry 326 for its operation. Implantable housing 324 includes a wall forming a chamber to house electronic circuitry 326 and power source 328. In the illustrated embodiment, electrodes 322A-B are each electrically coupled to electronic circuitry 326 and passing through the wall of implantable housing 324 at one of the opposite ends of implantable housing 324. In another embodiment, one or both of electrodes 322A-B are each electrically coupled to electronic circuitry 326 through a lead. In various embodiments, electrodes 322A-B function as a pair of stimulation electrodes for delivering neurostimulation and/or a pair of sensing electrodes for sensing a physiologic signal.

In one embodiment, implantable medical device 310 includes a BION® microstimulator (Advanced Bionics Corporation, a company of Boston Scientific Corporation, Sylmar, Calif., U.S.A.). The BION® microstimulator is discussed in, for example, U.S. Pat. Nos. 5,193,539; 5,193,540; 5,312,439; 5,324,316; 5,405,367; 6,051,017; and 6,185,452, which are incorporated by reference herein in their entireties. Implantable housing 324 is in the form of a capsule in an approximately cylindrical elongate shape. In various embodiments, as discussed below, positioning and/or anchoring devices are coupled to implantable housing 324 directly and/or via one or both of electrodes 322A-B to form an implantable medical device with an fixation and/or positioning mechanism.

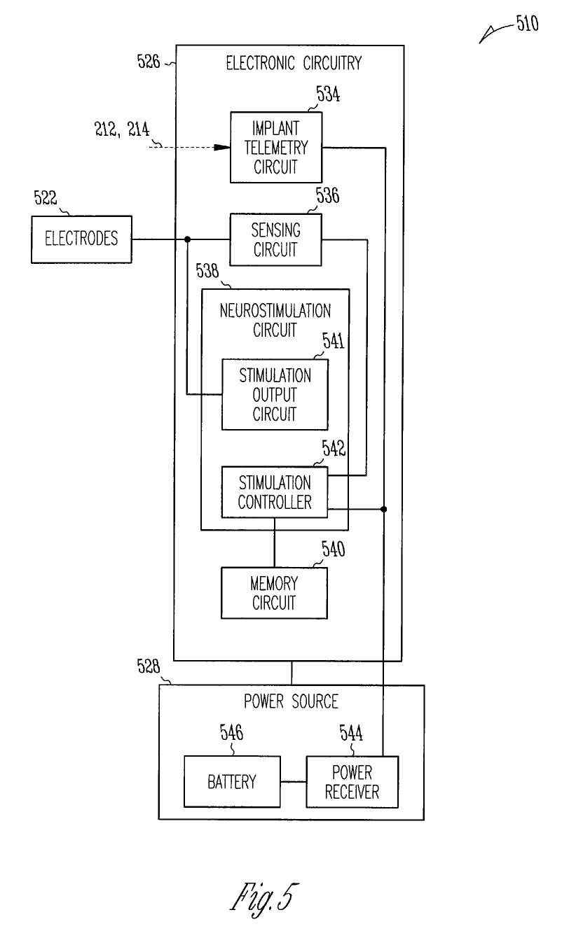

FIG. 4 is an illustration of an embodiment of an implantable medical device 410 showing its dimensions. Implantable medical device 410 represents the core of an implantable medical device with the fixation and/or positioning mechanism. In one embodiment, implantable medical device 410 represents implantable medical device 310 as illustrated in FIG. 3 (i.e., with electrodes). In another embodiment, implantable medical device 410 represents implantable housing 324 with electronic circuitry 326 and power source 328 housed therein (i.e., without electrodes).

In the illustrated embodiment, implantable medical device 410 has an approximately cylindrical elongate shape with a longitudinal axis 430. In one embodiment, implantable medical device 410 has a length 431 between approximately 5 mm and 30 mm and a diameter 432 between approximately 2 mm and 10 mm.

In one embodiment, implantable medical device 410 is configured to be injected through a hollow injection device having an end configured to reach a stimulation target region in body 102. Examples of the hollow injection device include a hollow needle and a hollow catheter.

FIG. 5 is a block diagram illustrating an embodiment of a circuit 510, which represents an example of a circuit of implantable medical device 310. Circuit 510 includes electrodes 522, electronic circuitry 526, and power source 528.

Electrodes 522 include at least a pair of electrodes allowing for delivery of neurostimulation and/or physiologic signal sensing, such as electrodes 322A-B. In one embodiment, electrodes 522 include more than two electrodes from which a set of two or more electrodes is selected at a time to deliver the neurostimulation. In one embodiment, electrodes 500 include multiple electrodes configured and arranged to allow for selective neural activation and/or optimization of neurostimulation parameters.

Electronic circuitry 526 is an embodiment of electronic circuitry 326. In the illustrated embodiment, electronic circuitry 526 includes an implant telemetry circuit 534, a sensing circuit 536, a neurostimulation circuit 538, and a memory circuit 540. In various embodiments, electronic circuitry 526 is capable of performing one or both of sensing and neurostimulation delivery functions. In another embodiment, circuit 510 is a circuit of a miniature implantable sensing device and includes at least implant telemetry circuit 534, and sensing circuit 536. In another embodiment, circuit 510 is the circuit of a miniature implantable neurostimulator and includes implant telemetry circuit 534, neurostimulation circuit 538, and memory circuit 540.

Implant telemetry circuit 534 allows circuit 510 to communicate with external system 220 via telemetry link 214 and/or implantable medical device 210 via telemetry link 212. Sensing circuit 536 senses a physiologic signal such as a neural signal or a cardiac signal through electrodes 522. Neurostimulation circuit 538 includes a stimulation output circuit 541 and a stimulation controller 542. Stimulation output circuit 541 delivers the neurostimulation to body 102 through electrodes 522. In one embodiment, stimulation output circuit 541 includes a pulse output circuit that delivers electrical stimulation pulses. In other embodiments, stimulation output circuit 541 includes a light emitter to deliver light stimulation, an ultrasonic transducer to deliver ultrasonic stimulation, or a magnetic field generator to deliver magnetic stimulation. Stimulation controller 542 controls the delivery of the neurostimulation by executing a stimulation algorithm.

In one embodiment, the stimulation algorithm executed by stimulation controller is for modulating a cardiovascular function. For example, the stimulation algorithm is defined by a plurality of stimulation parameters selected to provide one or more of a cardiac remodeling control therapy, an anti-arrhythmia therapy, and an anti-hypertension therapy. In one embodiment, the stimulation algorithm includes stimulation parameters for control of delivery of the electrical stimulation pulses. Examples of the stimulation parameters for controlling the delivery of electrical stimulation pulses include pulse amplitude, pulse width, stimulation frequency (or inter-pulse interval), periodic dose, and duty cycle. The pulse amplitude and pulse width are selected to ensure that each pulse elicits an action potential in the target nerve. In one embodiment, the stimulation frequency is between approximately 0.1 and 200 Hz, with between approximately 1 and 30 Hz as a specific example for modulating cardiovascular functions. The periodic dose is a time interval during which a patient is treated with neurostimulation for each predetermined period. In one embodiment, the predetermined period is a day, and the periodic dose is a daily dose. The duty cycle is the duty cycle of the neurostimulation during the time interval during of the period dose. For example, if the patient is to receive a neurostimulation therapy for two hours each day, the periodic dose is 2 hours/day (or the daily dose is 2 hours). If the neurostimulation during those two hours is delivered intermittently with alternating on- and off-periods, the duty cycle is the ratio of the on-period to the sum of the on-period and the off-period. In one embodiment, the daily dose is between approximately 0.5 and 24 hours. In one embodiment, the duty cycle is between approximately 10 and 50%. The on-period is between approximately 10 and 120 seconds, and the off-period is between approximately 50 and 120 seconds. Memory circuit 540 stores the stimulation algorithm including the stimulation parameters. In one embodiment, memory circuit 540 also stores the history of delivery of the neurostimulation.

Power source 528 is a specific embodiment of power source 328. In the illustrated embodiment, power source 528 includes a power receiver 544 and a battery 546. In one embodiment, battery 546 is a rechargeable battery. Power receiver 544 receives inductively transmitted or acoustically transmitted power via telemetry link 214 and converts the received power to direct-current (DC) power to supply electronic circuitry 526 and/or recharge rechargeable battery 546. In another embodiment, power source 528 includes power receiver 544 but not a battery, and the power is transmitted via telemetry link 214 during delivery of the neurostimulation therapy. Such a device may be suitable, for example, when the periodic dose is low. In another embodiment, power source 528 includes a non-rechargeable battery 546, for example, when a short-term neurostimulation therapy is intended.

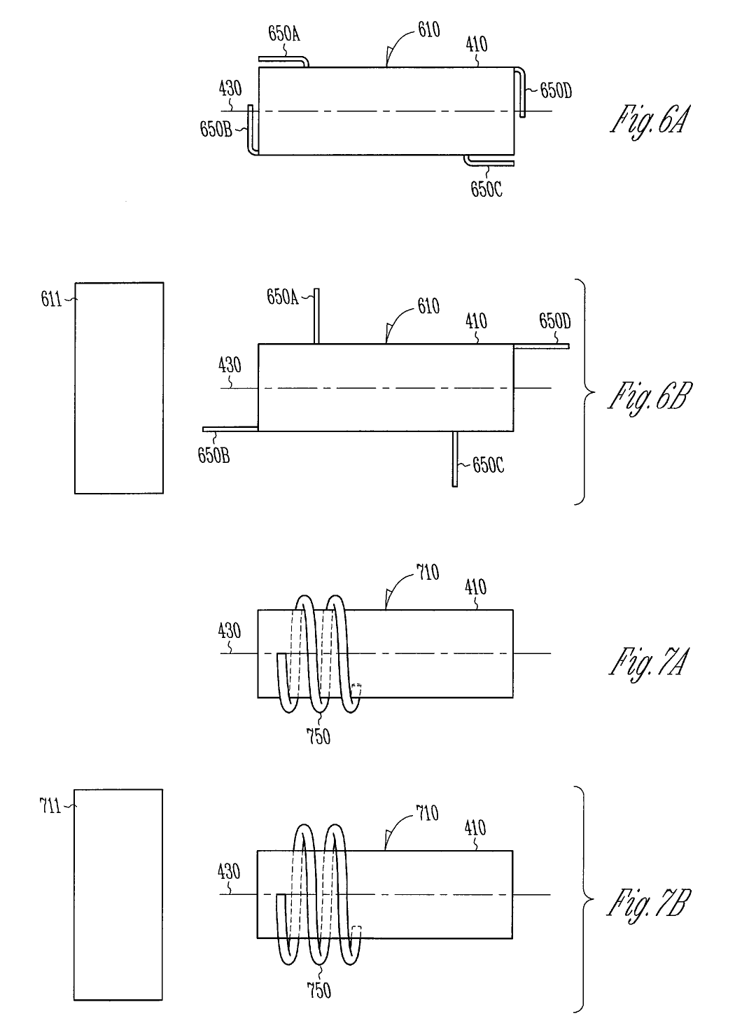

FIGS. 6-12 illustrate various embodiments of implantable medical devices each include positioning and/or fixation elements incorporated into implantable medical device 410. In FIGS. 6-9 and 11, implantable medical device 610, 710, 810, and 1100 each include an externally activated anchoring system. The externally activated anchoring system is incorporated into implantable medical device 410 by coupling to its implantable capsule, directly or via one or more electrodes, and includes one or more externally activated anchoring devices each switch from a substantially non-anchoring state to a substantially anchoring state in response to an externally applied energy. The externally applied energy is generated from an external anchoring activator being an embodiment of external device 111. To activate the externally activated anchoring system, the external anchoring activator is to be operably positioned with respect to an implanted medical device with the externally activated anchoring system. In one embodiment, at least one of the one or more externally activated anchoring devices is electrically connected to electronic circuitry 326 to function as an electrode for sensing and/or delivering neurostimulation.

In one embodiment, externally activated anchoring system includes one or more magnetically activated anchoring devices that switch from the substantially non-anchoring state to the substantially anchoring state in response to an externally applied magnetic field. The external anchoring activator is a magnetic field generator, such as a magnet.

In another embodiment, externally activated anchoring system includes one or more electrically activated anchoring devices that switch from the substantially non-anchoring state to the substantially anchoring state in response to an externally generated electric field. The external anchoring activator is an electric field generator, such as a voltage or current source connected to paddle electrodes configured for contacting the surface of body 102.

In another embodiment, externally activated anchoring system includes one or more electromagnetically activated anchoring devices that switch from the substantially non-anchoring state to the substantially anchoring state in response to an externally generated electromagnetic field. The external anchoring activator is an electromagnetic signal generator, such as radio frequency signal generator with a coil/antenna configured to be held on or near body 102.

In another embodiment, externally activated anchoring system includes one or more thermally activated anchoring devices that switch from the substantially non-anchoring state to the substantially anchoring state in response to thermal energy. In one embodiment, temperature of body 102 provides the thermal energy, such that the external anchoring activator is not necessary. In another embodiment, an external heating device functions as the external anchoring activator. In a specific embodiment, the external heating device is a heat pad configured for contacting the surface of body 102. In another specific embodiment, the external heating device is an electromagnetic signal generator suitable for heating portions of body tissue or portions of the anchoring system using electrical current induced by the electromagnetic signal.

The number, orientation, and distribution of the externally activated anchoring devices show in FIGS. 6-8 and 11 are for illustrative purpose only. In various embodiments, implantable medical device 610, 710, 810, and 1100 each include one or more externally activated anchoring devices distributed to achieve desirable fixation purposes. The number, orientation, and location of each externally activated anchoring device on each of implantable medical device 610, 710, 810, and 1100 depend on the structure of implantable medical device 410 and/or the anatomical structure of the intended implantation site.

FIGS. 6A-B are illustrations of an embodiment of implantable medical device 610 including implantable medical device 410 and externally activated anchoring devices 650A-D. Anchoring devices 650A-D each include a tine including an elongate body having an end coupled to implantable medical device 410. In the substantially non-anchoring state, the tine lies over a surface of implantable medical device 410, as illustrated in FIG. 6A. In the substantially anchoring state, the tine projects from the surface of implantable medical device 410, as illustrated in FIG. 6B. In the illustrated embodiment, the tines of anchoring devices 650A-D extend in directions that are approximately parallel or perpendicular to longitudinal axis 430. In other embodiments, the tines of anchoring devices 650A-D extend in any one or more directions to provide suitable anchoring function. In one embodiment, one or more of the tines are electrically connected to electronic circuitry 326 to function as one or more electrodes. In one embodiment, the tine includes a shape memory alloy. One example of such shape memory alloy is a nickel-titanium alloy such as Nitinol. Anchoring devices 650A-D switch from the substantially non-anchoring state to the substantially anchoring state when the tines are heated using an external anchoring activator 611 held in the vicinity of implantable medical device 610. External anchoring activator 611 is an embodiment of external device 111. In one embodiment, external anchoring activator 611 includes a heat source to heat the tissue surrounding the implanted implantable medical device 610, thereby heating the tines, causing them to switch from the state as illustrated in FIG. 6A to the state as illustrated in FIG. 6B. In another embodiment, the body temperature heats the tines when they are in the body, causing them to switch from the state as illustrated in FIG. 6A to the state as illustrated in FIG. 6B, without the need for external anchoring activator 611.

FIGS. 7A-B are illustrations of an embodiment of implantable medical device 710 including implantable medical device 410 and an externally activated anchoring device 750. Anchoring device 750 includes a coil coupled to implantable medical device 410. The coil is approximately coaxially wound over implantable medical device 410. In the substantially non-anchoring state, the coil has a diameter slightly bigger than the diameter of implantable medical device 410, as illustrated in FIG. 7A. In the substantially anchoring state, the coil has another diameter substantially bigger than the diameter of implantable medical device 410, as illustrated in FIG. 7B. In one embodiment, at least a portion of the coil is electrically coupled to electronic circuitry 326 to function as an electrode. In one embodiment, the coil includes a shape memory alloy. One example of such shape memory alloy is a nickel-titanium alloy such as Nitinol. Anchoring device 750 switches from the substantially non-anchoring state to the substantially anchoring state when the tines are heated using an external anchoring activator 711 held in the vicinity of implantable medical device 710. External anchoring activator 711 is an embodiment of external device 111. In one embodiment, external anchoring activator 711 includes a heat source to heat the tissue surrounding the implanted implantable medical device 710, thereby heating the coil, causing it to switch from the state as illustrated in FIG. 7A to the state as illustrated in FIG. 7B. In another embodiment, external anchoring activator 711 includes an electromagnetic signal generator to generate an electromagnetic signal for inducing a current in the coil, which is closed circuited. This generates heat in the coil, which causes the coil to switch from the state as illustrated in FIG. 7A to the state as illustrated in FIG. 7B. In another embodiment, the body temperature heats the coil when the coil is in the body, causing the coil to switch from the state as illustrated in FIG. 7A to the state as illustrated in FIG. 7B, without using external anchoring activator 711.

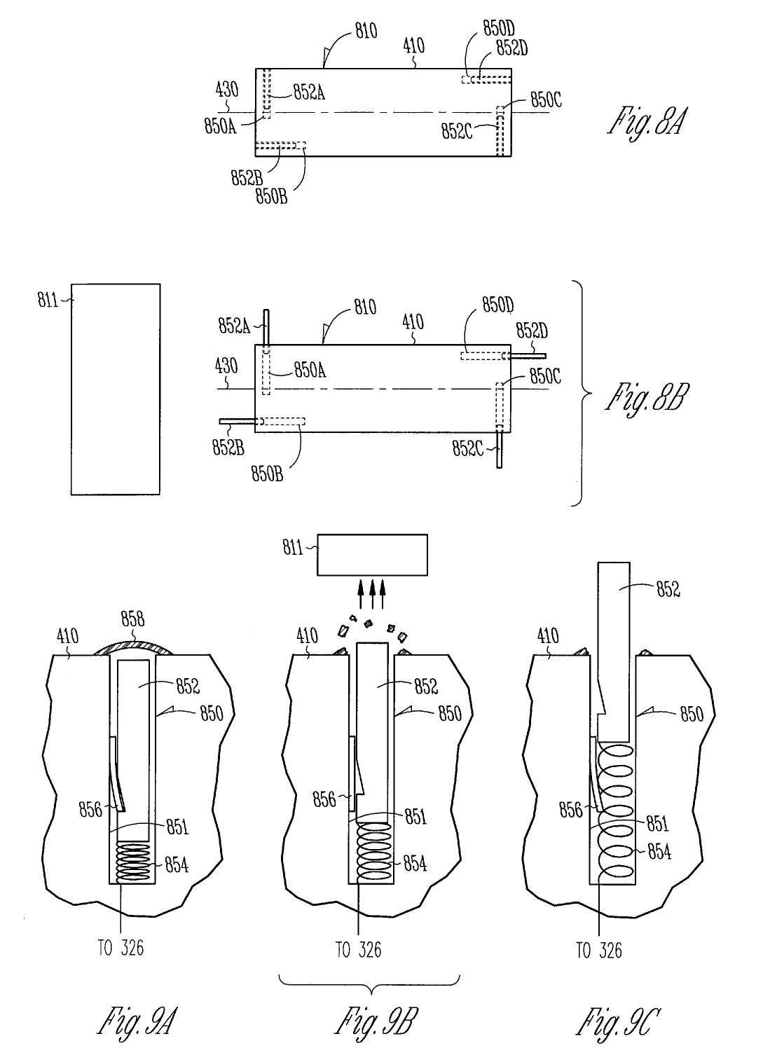

FIGS. 8A-B are illustrations of an embodiment of implantable medical device 810 and externally activated anchoring devices 850A-D. Anchoring devices 850A-D include tines 852A-D. In the substantially non-anchoring state, tines 852A-D are retained within implantable medical device 410, as illustrated in FIG. 8A. In the substantially anchoring state, tines 852A-D extend from implantable medical device 410, as illustrated in FIG. 8B. In the illustrated embodiment, 852A-D extend in directions that are approximately parallel or perpendicular to longitudinal axis 430. In other embodiments, 852A-D extend in any one or more directions to provide suitable anchoring function. In one embodiment, one or more of tines 852A-D are electrically connected to electronic circuitry 326 to function as one or more electrodes. Anchoring devices 850A-D switch from the substantially non-anchoring state to the substantially anchoring state in response to a magnetic field generated from an external anchoring activator 811 held in the vicinity of implantable medical device 810. External anchoring activator 811 is an embodiment of external device 111 and includes a magnetic field generator such as a magnet. Tines 852A-D shoot out from implantable medical device 410 in response to the application of the magnetic field.

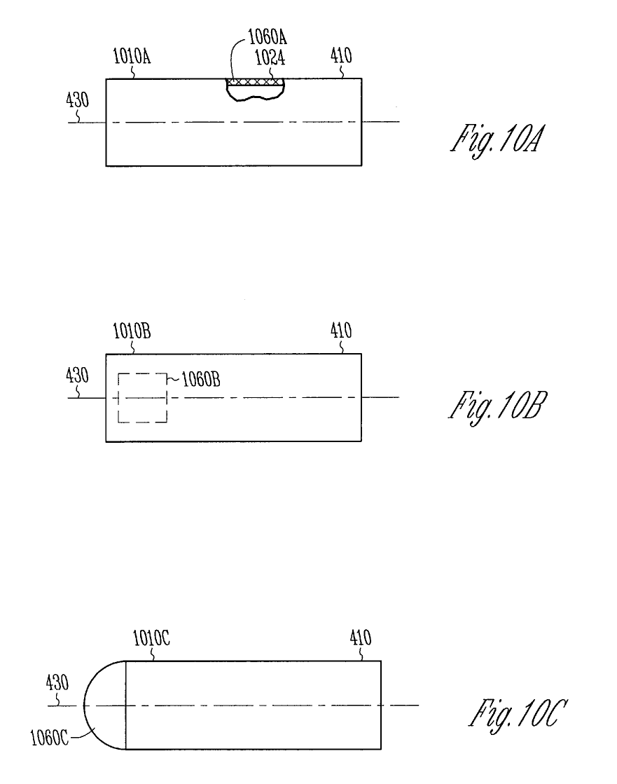

FIGS. 9A-C are detailed illustrations of an embodiment of an externally activated anchoring device 850, which represents a specific embodiment of one of externally activated anchoring devices 850A-D. Anchoring device 850 includes a well 851, a tine 852, a spring 854, and an activation switch 856. Well 851 is formed in the implantable housing and/or one of the electrodes of implantable medical device 410. Tine 852 represents a specific embodiment of one of tines 852A-D. Spring 854 connects tine 852 to implantable medical device 410. In the substantially non-anchoring state, spring 854 is in a restrained state forced by activation switch 856, thereby keeping tine 852 substantially within well 851. In response to the application of the magnetic field, activation switch 856 releases spring 854 from the restrained state. Tine 852 shoots out of well 851 to enter tissue of body 102 as spring 854 transitions from the restrained state to the unrestrained state.

In the illustrated embodiment, activation switch 856 includes a magnetic reed. FIG. 9A illustrates anchoring device 850 in the substantially non-anchoring state, with the magnetic reed applying a restraining force upon spring 854. FIG. 9B illustrates the transitioning of anchoring device 850 from the substantially non-anchoring state to the substantially anchoring state in response to the magnetic field applied using external anchoring activator 811. The magnetic reed no longer provides the restraining force. FIG. 9C illustrates anchoring device 850 in the substantially anchoring state, with spring 854 in its unrestrained state.

In one embodiment, as illustrated in FIG. 9A, the opening of well 851 is covered by a seal 858 to prevent fluids from entering while implantable medical device 410 is being positioned. In the illustrated embodiment, seal 858 is in the form of a protective dome. In other embodiments, seal 858 is in any form that prevents fluids from entering well 851 during implantation. In one embodiment, as illustrated in FIG. 9B, seal 858 is configured to allow tine 852 to break through it following the release of spring 854 from the restrained state. In another embodiment, seal 858 dissolves soon (such as between 5 and 12 minutes) after being deployed in the body. The magnetic field is then applied to release spring 854 from the restrained state. Seal 858 is made of a bio-absorbable material. An example of such material is polyglycolic acid (PGA).

FIGS. 10A-C are each an illustration of an embodiment of a magnetically positionable implantable medical device. The magnetically positionable implantable medical device includes implantable medical device 410 and a positioning element. The positioning element includes a magnetized or ferromagnetic material to allow the implantable medical device to be positioned in body 102 using magnetic attraction during its implantation and/or to be fixed to a position in body 102 using magnetic attraction following its implantation.

FIG. 10A is an illustration of an embodiment of a magnetically positionable implantable medical device 1010A. Implantable medical device 1010A includes implantable medical device 410 with an implantable housing 1024, which represents a specific embodiment of implantable housing 324. A positioning element 1060A is incorporated into implantable housing 1024. In one embodiment, implantable housing 1024 includes a magnetized or ferromagnetic material forming positioning element 1060A.

FIG. 10B is an illustration of an embodiment of a magnetically positionable implantable medical device 1010B. Implantable medical device 1010B includes implantable medical device 410 and a positioning element 1060B housed within implantable medical device 410. In one embodiment, positioning element 1060B is a magnetized or ferromagnetic structure dedicated for positioning purposes. In another embodiment, positioning element 1060B is part of electronic circuitry 326 or power source 328 that is constructed using a magnetized or ferromagnetic material.

FIG. 10C is an illustration of an embodiment of a magnetically positionable implantable medical device 1010C. Implantable medical device 1010C includes implantable medical device 410 and a positioning element 1060C affixed onto implantable medical device 410. In the illustrated embodiment, positioning element is a magnetized or ferromagnetic nosecone attached to an end of implantable medical device 410.

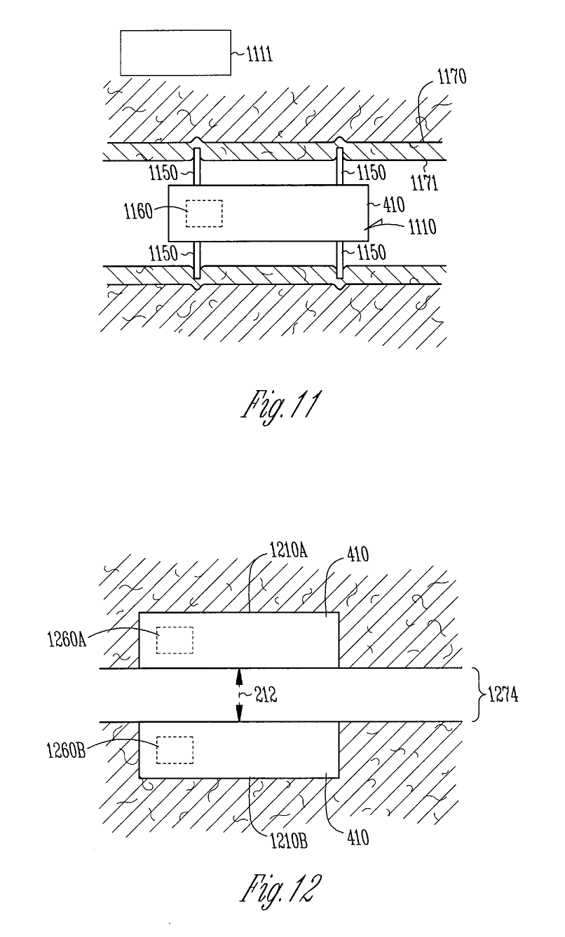

FIG. 11 is an illustration of an embodiment of an implantable medical device 1110 placed in a lumen 1171 of a tubular structure 1170 of body 102. Implantable medical device 1110 includes implantable medical device 410, a positioning element 1160, and externally activated anchoring devices 1150. The configuration of positioning element 1160 and externally activated anchoring devices 1150 as shown in FIG. 11 are for illustrative purposes only. In various embodiments, positioning element 1160 takes the form of any of positioning elements 1060A-C as illustrated in FIG. 10, and externally activated anchoring devices 1150 takes the form of any of anchoring devices 650A-D, 750, and 850A-D as illustrated in FIG. 6-9.

An external device 1111 includes one or both of an external positioning device and an external anchoring activator. In one embodiment, external device 1111 includes an integrated external positioning device and anchoring activator. In a specific embodiment, a magnetic field generator, such as a magnet, functions as both the external positioning device and the external anchoring activator. In another embodiment, external device 1111 applies different forms of energy are position implantable medical device 1110 and activate externally activated anchoring devices 1150, to allow the anchoring to occur after implantable medical device 1110 is positioned in a desirable location in lumen 1171. In a specific embodiment, external device 1111 includes physically separate external positioning device and external anchoring activator.

In one embodiment, tubular structure 1170 is a blood vessel. Implantable medical device 1110 with externally activated anchoring devices 1150 in a substantially non-anchoring state is injected into lumen 1171. After being injected into the blood vessel, the position of implantable medical device 1110 is adjustable using external device 1111. After implantable medical device 1110 is in the intended position, anchoring devices 1150 are activated by external device 1111 to transition from the substantially non-anchoring state to the substantially anchoring state, thereby preventing implantable medical device 1110 from moving along the blood vessel.

FIG. 12 is an illustration of an embodiment of a system of a plurality of magnetically positionable implantable medical devices. For illustrative purposes, a dual-device system including magnetically positionable implantable medical devices 1210A and 1210B is shown. In various embodiments, the system includes two or more magnetically positionable implantable medical devices such as implantable medical devices 1210A and 1210B. Implantable medical devices 1210A and 1210B each represent one of implantable medical devices 1010A-C as illustrated in FIG. 10. Positioning elements 1260A-B are incorporated into implantable medical devices 1210A-B and each represent one of positioning elements 1060A-C.

Implantable medical devices 1210A-B are to be placed in the vicinity of each other such that they are magnetically attracted to each other after being implanted in body 102. The magnetic attraction is created by positioning elements 1260A-B and has a strength suitable for preventing implantable medical devices 1210A-B from migrating in body 102 without causing intolerable pressure on any tissue in body 102. In one embodiment, as illustrated in FIG. 12, implantable medical devices 1210A-B are neurostimulators placed on approximately opposite side of a nerve 1274 such that the magnetic attraction maintains their positions relative to nerve 1274 to allow, for example, consistent neural response to the neurostimulation.

In one embodiment, the magnetically positionable implantable medical devices are grouped in a way allowing for selective stimulation of one or more targets such as one or more nerves in a nerve bundle. The stimulation energy is directed by programming stimulation parameters of each of the magnetically positionable implantable medical devices. For example, when the neurostimulation is delivered in the form of electrical pulses, the direction of electrical current is steered by controlling the pulse amplitude in each of the magnetically positionable implantable medical devices.

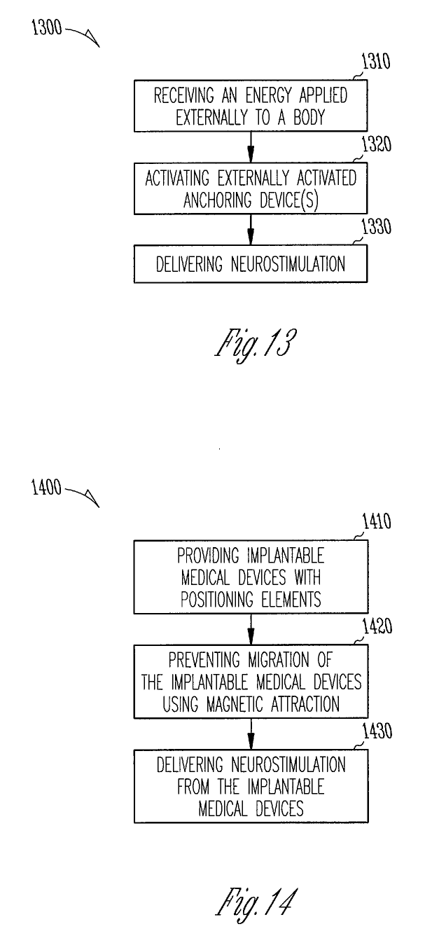

FIG. 13 is a flow chart illustrating a method 1300 for externally activating an anchoring system of an implantable medical device. Examples of such an implantable medical device include implantable medical devices 610, 710, 810, and 1110.

An energy externally applied to the body of a patient is received by the anchoring system at 1310. Examples of the energy include energy associated with a magnetic field, energy associated with an electric field, energy of an electromagnetic signal, and thermal energy. The energy is generated from an external device and received when the external device is held on or near the body over the implantable medical device.

In response to the reception of the externally applied energy, one or more externally activated anchoring devices of the anchoring system are activated at 1320. The activation of each externally activated anchoring device includes its transitioning from a substantially non-anchoring state to a substantially anchoring state. In one embodiment, this includes releasing an anchoring component of the externally activated anchoring device from a restrained (forced) state to an unrestrained (natural) state. In one embodiment, each externally activated anchoring device includes an anchoring component constructed with a shape memory alloy. The anchoring component transitions from the substantially non-anchoring state to the substantially anchoring state when the shape memory alloy is heated. The externally activated anchoring device is activated by heating the component directly or through tissue heating using thermal or electromagnetic energy. In another embodiment, each externally activated anchoring device includes an anchoring component coupled to a spring that in its restrained state keeps the anchoring component substantially within the implantable medical device. The anchoring component is delivered to its anchoring position when the spring is released from its restrained state. The spring is kept in its restrained state using a magnetic reed. The externally activated anchoring device is activated by applying a magnetic field to the magnet reed to release the spring.

After the anchoring system is activated to fix the implantable medical device to a position in or near a stimulation target in the body, neurostimulation is delivered at 1330. In one embodiment, the delivery of the neurostimulation is controlled by executing a neurostimulation algorithm for modulating cardiovascular function. In one embodiment, in addition to or instead of delivering the neurostimulation, a physiologic signal is sensed, and/or another therapy is delivered, by the implantable medical device.

In another embodiment, the thermal energy if the body of the patient replaces the energy externally applied to the body at 1310. This allows the one or more externally activated anchoring devices of the anchoring system to be activated automatically following the implantation of the implantable medical device.

FIG. 14 is a flow chart illustrating a method 1400 for magnetically coupling a plurality of implantable medical devices. The magnetic coupling between the implantable medical devices prevents each of the implantable medical devices from migrating from an intended region after being implanted in the body of a patient.

The implantable medical devices are provided with positioning elements at 1410. The positioning elements each include a magnetized or ferromagnetic material. In various embodiments, a positioning element is externally affixed onto the housing of each implantable medical device, incorporated into the housing, or contained within the housing. Examples of such implantable medical devices include implantable medical devices 1010A-C.

After being implanted, migration of the implantable medical devices is prevented using magnetic attraction between the positioning elements at 1420. At least one of the implantable medical devices has a positioning element including a magnetized material.

Neurostimulation is delivered from the implantable medical devices at 1430. In one embodiment, the implantable medical devices communicate with each other via telemetry for coordinated operation including neurostimulation and/or physiologic signal sensing. In one embodiment, the direction of an electrical current of the neurostimulation is controlled by programming stimulation parameters of each of the implantable medical devices. In one embodiment, the delivery of the neurostimulation is controlled by executing a neurostimulation algorithm for modulating cardiovascular function. In one embodiment, in addition to or instead of delivering the neurostimulation, a physiologic signal is sensed, and/or another therapy is delivered, by the implantable medical device.

It is to be understood that the above detailed description is intended to be illustrative, and not restrictive. For example, the anchoring and positioning devices according to the present subject matter may have various specific configurations that deviate from the specific examples discussed in this document and are applicable to implantable medical devices having shapes and functions not limited to the specific examples discussed in this document. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system, comprising:
an implantable medical device including:
electronic circuitry;
an implantable housing including a wall forming a chamber to house the electronic circuitry; and
one or more externally activated anchoring devices coupled to the implantable housing, the one or more externally activated anchoring devices each configured to switch from a substantially non-anchoring state to a substantially anchoring state in response to an externally applied energy; and
an external anchoring activator configured to be operably positioned with respect to the implantable medical device to provide the externally applied energy.

2. The system of claim 1, wherein the implantable housing has an approximately cylindrical elongate shape, the electronic circuitry comprises a stimulation output circuit adapted to deliver the neurostimulation and a stimulation controller adapted to control the delivery of the neurostimulation, and the implantable medical device comprises electrodes each coupled to the stimulation output circuit.

3. The system of claim 2, wherein at least one of the electrodes is incorporated into one of the one or more externally activated anchoring devices.

4. The system of claim 1, wherein the external anchoring activator comprises a magnet.

5. The system of claim 1, wherein the one or more externally activated anchoring devices each comprise a tine including an elongate body having an end coupled to the implantable housing, the tine in a restrained state lying over a surface of the implantable housing and in a unrestrained state projecting from the surface of the implantable housing.

6. The system of claim 5, wherein the tine comprises a shape memory alloy and is configured to switch from the restrained state to the unrestrained state in response to heating of the shape memory alloy.

7. The system of claim 1, wherein the one or more externally activated anchoring devices each comprise a coil formed by a coiled wire coupled to the implantable housing, the coil having a first diameter in a restrained state and a second diameter in an unrestrained state, the second diameter substantially greater than the first diameter.

8. The system of claim 7, wherein the coil comprises a shape memory alloy and is configured to switch from the first diameter to the second diameter in response to heating of the shape memory alloy.

9. The system of claim 1, wherein the one or more externally activated anchoring devices each comprise an activation switch adapted to switch the each of the one or more externally activated anchoring devices from the substantially non-anchoring state to the substantially anchoring state in response to an externally applied magnetic energy.

10. The system of claim 9, wherein the one or more externally activated anchoring devices each comprise a tine coupled to the activation switch, the tine coupled to a spring coupled to the implantable housing and configured to deliver the tine from the non-anchoring state to the anchoring state when the spring is released from a restrained state to an unrestrained state, and the activation switch comprises a magnetic reed configured to release the spring from the restrained state to the unrestrained state in response to the externally applied magnetic energy.

11. The system of claim 1, wherein the implantable medical device is a magnetically positionable implantable medical device comprising a positioning element including a magnetized or ferromagnetic material.

12. An implantable system for use in a living body, the system comprising:
a plurality of magnetically positionable implantable medical devices adapted to be magnetically attracted to each other after being placed in the body, the magnetically positionable implantable medical devices including positioning elements configured to prevent the magnetically positionable implantable medical devices from migrating in the body using magnetic attraction.

13. The system of claim 12, wherein the implantable medical devices each comprise electronic circuitry and an implantable housing configured to house the electronic circuitry, the electronic circuitry comprising a stimulation output circuit adapted to deliver the neurostimulation and a stimulation controller adapted to control the delivery of the neurostimulation.

14. The system of claim 13, wherein the positioning elements comprise one or more of a magnetized material and a ferromagnetic material.

15. The system of claim 13, wherein the implantable housing comprises one of the magnetized material and the ferromagnetic material.

16. A method for operating an implantable medical device in a living body, the method comprising:
receiving an energy applied externally to the body; and
switching one or more externally activated anchoring devices of the implantable medical device from a substantially non-anchoring state to a substantially anchoring state in response to the received energy.

17. The method of claim 16, further comprising delivering neurostimulation from the implantable medical device.

18. The method of claim 17, wherein delivering the neurostimulation comprises delivering the neurostimulation through at least one electrode incorporated into the one or more externally activated anchoring devices.

19. The method of claim 16, wherein receiving the energy comprises receiving one of an energy associated with a magnetic field, an energy associated with an electric field, an energy of an electromagnetic signal, and a thermal energy.

20. The method of claim 16, wherein switching the one or more externally activated anchoring devices from the substantially non-anchoring state to the substantially anchoring state comprises raising temperature of an anchoring structure including a shape memory alloy.

21. The method of claim 20, wherein receiving the energy comprises receiving a thermal energy from one of an external heating device and an external electromagnetic signal generator.

22. The method of claim 16, wherein switching the one or more externally activated anchoring devices from the substantially non-anchoring state to the substantially anchoring state comprises using one or more magnetic reed switches.

23. The method of claim 22, wherein switching the one or more externally activated anchoring devices from the substantially non-anchoring state to the substantially anchoring state comprises releasing a spring from a restrained state using the one or more magnetic reed switches.

24. A method for operating a system of a plurality of implantable medical devices in a living body, the method comprising:
providing each of the implantable medical devices in the body with a positioning element including a magnetized or ferromagnetic material; and
preventing migration of the implantable medical devices in the body using magnetic attraction between the implantable medical devices.

25. The method of claim 24, further comprising delivering neurostimulation from the implantable medical devices.

26. The method of claim 25, further comprising steering an electrical current of the neurostimulation by programming stimulation parameters of each of the implantable medical devices.

27. The method of claim 25, wherein delivering the neurostimulation comprises executing a neurostimulation algorithm for modulating a cardiovascular function.
